(19) United States
(12) Patent Application Publication
(10) Pub. No.: US 2026/0271850 A1
(43) Pub. Date: Sep. 17, 2026

(54) METHOD FOR EFFICIENT AND RAPID PROPAGATION OF EMMENOPTERYS HENRYI

(71) Applicant: HUAZHONG AGRICULTURAL UNIVERSITY, Wuhan (CN)

(72) Inventors: Xueping SHI, Wuhan (CN); Bo ZHENG, Wuhan (CN); Shenghao WU, Wuhan (CN); Mei LIU, Wuhan (CN); Li YANG, Wuhan (CN); Shiyi YANG, Wuhan (CN); Zhixiang ZHOU, Wuhan (CN); Songzhu XIANG, Wuhan (CN); Qiang FU, Wuhan (CN)

(21) Appl. No.: 19/643,255

(22) Filed: Apr. 9, 2026

(30) Foreign Application Priority Data
Mar. 13, 2025 (CN) 202510295475.6

(51) Int. Cl. A01H 4/00

(52) U.S. Cl. CPC A01H 4/008; A01H 4/002

(57) ABSTRACT

A method for efficient and rapid propagation of Emmenopterys henryi includes obtaining sterile seedlings, inducing adventitious buds, proliferating adventitious buds, promoting elongation of adventitious buds, promoting rooting of adventitious buds, and hardening and transplanting tissue culture plantlets. Different plant hormone components are selected to prepare a culture system including an initiation medium, a differentiation medium, a proliferation medium, an elongation medium, and a rooting medium. The differentiation rate of adventitious buds of Emmenopterys henryi is as high as 81.7%, the proliferation coefficient of adventitious buds is 4.7, the proportion of adventitious buds with a height greater than 5 mm after proliferation culture reaches 72.5%, and the rooting rate is 77.8%. When the tissue culture plantlets are transplanted into a mixed substrate, the survival rate reaches 100%.

### CROSS-REFERENCE TO RELATED APPLICATIONS

The present application claims priority to Chinese Patent Application No. 202510295475.6, filed on Mar. 13, 2025, the entire disclosure of which is incorporated herein by reference.

### TECHNICAL FIELD

The present disclosure relates to the technical field of plant tissue culture, and in particular to a method for the efficient and rapid propagation of Emmenopterys henryi.

### BACKGROUND

Emmenopterys henryi is characterized by a straight trunk, making it suitable for timber production. Its fine and elastic fibers are ideal for manufacturing stencil paper and rayon. Its roots and bark have medicinal properties with applications in treating symptoms such as vomiting. Additionally, with its graceful branches and beautiful flowers, it serves as an excellent ornamental tree species for landscaping. However, the wild population of Emmenopterys henryi is extremely small (Xu Xiaoyu et al., 2002; Yang Kaijun et al., 2007; Fang Yuanping et al., 2007). Due to habitat destruction, over-exploitation, and a low natural regeneration rate, its population has declined sharply. Although the winged seeds of Emmenopterys henryi are small and light, allowing for wind dispersal (Li Tiehua et al., 2004), their germination rate in natural habitats is low due to limitations in light and humidity, and seedlings are rarely seen in forests. Consequently, Emmenopterys henryi faces the risk of extinction in the wild (Gan et al., 2006).

Research on Emmenopterys henryi has mostly focused on population structure and dynamics. Although such species is distributed across fourteen provinces and regions in central and eastern China, its populations are sparse and only grow sporadically in high-altitude, high-humidity mountainous areas (Yang Kaijun and Zhang Xiaoping, 2007) and show a trend of degradation in most areas. In the wild, the renewal of Emmenopterys henryi populations mainly relies on population reproduction, seed germination, and seedling bank supplementation. The trees do not reach their initial flowering stage until they are 20-30 years old (Xu Xiaoyu et al., 2002; Guo Lianjin et al., 2015). Furthermore, the abortion rates during the flowering and fruiting stages are extremely high (reaching 80% and 60%, respectively), resulting in a very low seed setting rate (Guo Lianjin et al., 2011). The seeds of Emmenopterys henryi have a life cycle of only one year (Li Zhongyue and Ban Qing, 1995) and are sensitive to light (Li Tiehua et al., 2004), and thus a suitable light environment is crucial for seed germination and seedling establishment (Guo Lianjin, 2021). In the wild, population renewal can also occur through asexual reproduction methods such as cutting propagation, but the highest rooting rate for cuttings and root burying is only 68.61% (Wan Jun et al., 2017). Tissue culture technology, as an efficient method of asexual reproduction, can produce a large number of high-quality Emmenopterys henryi seedlings in a short time at a relatively low cost, offering an effective way to alleviate its endangered status.

Explants such as embryos, stem segments, leaves, and buds of Emmenopterys henryi can be used for in vitro regeneration and propagation under certain tissue culture conditions, through somatic embryogenesis or direct/indirect organogenesis pathways (Ji Feiteng et al., 2005; Xiong Dan et al., 2007; Hong Senrong and Yin Minghua, 2010; Su Jing et al., 2008; Peng Xiang et al., 2018; Li Jinrong et al., 2015). For instance, Ji Feiteng et al. (2005) induced callus from leaves, then induced somatic embryos through suspension culture of the callus, and finally obtained plantlets through the germination of somatic embryos. However, this process involves many steps, and lacks support from specific experimental data. Xiong Dan et al. (2007) studied the direct induction of adventitious buds from Emmenopterys henryi leaves. Although the induction rate was high, the proportion of adventitious buds with a height≥5 mm was only about 43% after 40 days of culture. Su Jing et al. (2008) used Emmenopterys henryi leaves for adventitious bud induction, but the experimental data lacked replicates, resulting in low reliability, and no data on the height of the induced adventitious buds were provided. Hong Senrong and Yin Minghua (2010) studied the proliferation process of sterile seedlings but lacked research on processes such as adventitious bud induction, elongation, and rooting. Peng Xiang et al. (2018) used leaves and petioles to induce callus and then differentiate adventitious buds, which is multi-step and time-consuming.

The main problems existing in current research reports on the rapid propagation of Emmenopterys henryi via tissue culture are summarized as follows:
(1) The plant regeneration process requires a callus stage, which involves many steps and is time-consuming.
(2) Existing reports lack a systematic protocol of whole-process plant regeneration. Even where some studies cover various steps of regeneration and rapid propagation, issues such as difficulty in directly inducing adventitious buds, low proliferation rates of adventitious buds persist. Additionally, many experiments lack detailed data or sufficient replicates, making it impossible to judge the reliability of the results.
(3) The elongation rate of Emmenopterys henryi adventitious buds is slow, and most studies lack reports on adventitious bud height and elongation rate.

Therefore, there is an urgent need to overcome the deficiencies of the existing technology so as to realize the efficient and rapid propagation of Emmenopterys henryi.

### SUMMARY

The objective of the present disclosure is to provide a method for the efficient and rapid propagation of Emmenopterys henryi to address the problems existing in the prior art. The method provided by the present disclosure can cultivate high-quality Emmenopterys henryi saplings efficiently and rapidly, offering important technical guidance for rescuing Emmenopterys henryi from its endangered predicament.

To achieve the above objective, the present disclosure provides the following technical solution:
A method for efficient and rapid propagation of Emmenopterys henryi, comprising the following steps:
culturing a tender stem with buds from an Emmenopterys henryi seedling as an explant on an initiation medium to obtain a sterile seedling;
taking leaves of the sterile seedling and inducing differentiation of the leaves on a differentiation medium to obtain adventitious buds;
subjecting the adventitious buds to proliferation culture on a proliferation medium to obtain proliferated adventitious buds;
culturing the proliferated adventitious buds on an elongation medium to obtain elongated adventitious buds;
subjecting the elongated adventitious buds to rooting culture on a rooting medium to obtain tissue culture plantlets; and
transplanting the tissue culture plantlets into a mixed substrate for hardening.

Preferably, the initiation medium is Murashige and Skoog (MS) medium containing 2.0 mg/L 6-benzylaminopurine (6-BA), 2.0 mg/L kinetin (KT), and 0.1 mg/L 1-naphthaleneacetic acid (NAA).

Preferably, the differentiation medium is MS medium containing 1.0 mg/L 6-BA, 0.1 mg/L NAA, and 0.01 mg/L thidiazuron (TDZ).

Preferably, the proliferation medium is MS medium containing 3.0 mg/L 6-BA and 0.1 mg/L indole-3-butyric acid (IBA).

Preferably, the elongation medium is MS medium containing 3.0 mg/L 6-BA, 0.1 mg/L IBA, and 0.1 mg/L gibberellic acid (GA₃).

Preferably, prior to the elongation culture, the method further comprises a step of removing leaves from the proliferated adventitious buds and retaining only terminal buds. Preferably, the rooting medium is ½ MS medium containing 1.0 mg/L IBA.

Preferably, the mixed substrate is composed of peat soil, substrate soil, and vermiculite, wherein a mass ratio of the peat soil, the substrate soil, and the vermiculite is 3:3:1.

The present disclosure further provides a culture medium combination for Emmenopterys henryi, comprising an initiation medium, a differentiation medium, a proliferation medium, an elongation medium, and a rooting medium;
wherein the initiation medium is MS medium containing 2.0 mg/L 6-BA, 2.0 mg/L KT, and 0.1 mg/L NAA;
the differentiation medium is MS medium containing 1.0 mg/L 6-BA, 0.1 mg/L NAA, and 0.01 mg/L TDZ;
the proliferation medium is MS medium containing 3.0 mg/L 6-BA and 0.1 mg/L IBA;
the elongation medium is MS medium containing 3.0 mg/L 6-BA, 0.1 mg/L IBA, and 0.1 mg/L GA₃; and
the rooting medium is ½ MS medium containing 1.0 mg/L IBA.

The present disclosure further provides an application of the aforementioned culture medium combination in the propagation of Emmenopterys henryi.

The present disclosure discloses the following technical effects:
(1) In respect of optimizing the tissue culture rapid propagation technology for Emmenopterys henryi, the present disclosure systematically compared the efficiency of inducing adventitious buds from leaves using combinations of 6-BA and NAA versus combinations of 6-BA, NAA, and TDZ. By comparing 9 formulations of 6-BA and NAA combinations and 5 formulations of 6-BA, NAA, and TDZ combinations, the present disclosure screened out the optimal culture medium formula for direct adventitious bud induction: MS+1.0 mg/L 6-BA+0.1 mg/L NAA+0.01 mg/L TDZ+30 g/L sucrose+7.5 g/L agar. This formula achieved a direct adventitious bud induction rate of 81.7% from Emmenopterys henryi sterile seedling leaves, with 74.2% of the induced adventitious buds being clustered shoots, which is significantly superior to the prior art techniques.
(2) To improve the proliferation efficiency of Emmenopterys henryi, the present disclosure compared the effects of 6-BA and IBA combinations on adventitious bud proliferation, identifying the optimal proliferation medium formula as MS+2.0 mg/L 6-BA+0.1 mg/L IBA+30 g/L sucrose+7.5 g/L agar. On this medium, the proliferation coefficient reached 3.9, and the adventitious buds grew well. Furthermore, the effects of 6-BA concentrations on bud proliferation were investigated. It was found that when the concentration of 6-BA was raised to 3.0 mg/L, the proliferation coefficient increased to 4.7, and the proportion of adventitious buds with a height greater than 5 mm reached 72.5%. This is significantly higher than results published in the literature (Xiong Dan et al., 2007). This represents a major advancement in the rapid propagation technology for Emmenopterys henryi.
(3) Emmenopterys henryi exhibits a relatively slow growth rate. To promote the elongation of the adventitious buds, the present disclosure compared the effects of five different concentrations of GA₃. By adding GA₃ to the proliferation medium, the growth rate of adventitious bud height was significantly increased. Among these, the medium supplemented with 0.1 mg/L GA₃ resulted in the fastest height growth, which was significantly higher than that of the control and other concentration treatments (FIG. 2). This marks another major advancement in the rapid propagation technology for Emmenopterys henryi.

### BRIEF DESCRIPTION OF THE DRAWINGS

To more clearly illustrate the technical solutions in the embodiments of the present invention or in the prior art, the accompanying drawings required for the description of the embodiments are briefly introduced below. It is obvious that the drawings in the following description are only some embodiments of the present disclosure, and for those of ordinary skill in the art, other drawings can be obtained based on these drawings without creative efforts.

FIGS. 1A-G illustrate the efficient and rapid propagation process of Emmenopterys henryi, wherein:

FIG. 1A shows a sterile seedling obtained by culturing a terminal bud explant of Emmenopterys henryi in MS medium containing 2.0 mg/L 6-BA, 2.0 mg/L KT, and 0.1 mg/L NAA for 30 days (scale bar=1 cm);

FIG. 1B shows adventitious buds induced from leaves cultured in MS medium containing 0.1 mg/L NAA and 1.0 mg/L 6-BA for 30 days, with arrows pointing to the induced adventitious buds (scale bar=1 cm);

FIG. 1C shows adventitious buds induced from leaves cultured in MS medium containing 0.1 mg/L NAA, 1.0 mg/L 6-BA, and 0.01 mg/L TDZ for 30 days (scale bar=1 cm);

FIG. 1D is an enlarged view of the boxed area in FIG. 1C (scale bar=0.1 cm);

FIG. 1E shows adventitious buds after proliferation culture in MS medium containing 3.0 mg/L 6-BA and 0.1 mg/L IBA for 40 days (scale bar=1 cm);

FIG. 1F shows adventitious buds rooting in MS medium containing 1.0 mg/L IBA (scale bar=1 cm); and

FIG. 1G shows a rooted Emmenopterys henryi tissue culture plantlet (scale bar=1 cm).

FIG. 2 shows the effect of GA₃ on the elongation of Emmenopterys henryi adventitious buds, wherein the horizontal axis represents GA₃ concentration, and the vertical axis represents the height increase of adventitious buds after treatment with different concentrations of GA₃ for 10 days, 15 days, and 20 days. Different letters in the figure indicate significant differences (P<0.05).

### DETAILED DESCRIPTION OF THE EMBODIMENTS

Multiple exemplary embodiments of the present disclosure are now described in detail. This detailed description should not be construed as a limitation on the present disclosure but rather as a more detailed description of certain aspects, features, and embodiments of the present disclosure.

It should be understood that the terms used in the present disclosure are merely for describing particular embodiments and are not intended to limit the present disclosure. Additionally, for numerical ranges in the present disclosure, it should be understood that every intermediate value between the upper and lower limits of that range is also disclosed. Any stated value or intermediate value within a stated range, as well as any other stated value or every smaller range between intermediate values in said range, is also included within the present disclosure. The upper and lower limits of these smaller ranges may independently be included or excluded from the range.

Unless otherwise specified, all technical and scientific terms used herein have the same meaning as commonly understood by one of ordinary skill in the art to which the present disclosure belongs. Although only preferred methods and materials are described herein, any methods and materials similar or equivalent to those described herein can be used in the practice or testing of the present disclosure. All documents mentioned in this specification are incorporated by reference to disclose and describe the methods and/or materials associated with said documents. In case of conflict with any incorporated document, the content of this specification shall control.

Various improvements and changes can be made to the specific embodiments of the specification of the present disclosure without departing from the scope or spirit of the present disclosure, which is obvious to those skilled in the art. Other embodiments derived from the specification of the present disclosure are obvious to those skilled in the art. The specification and examples of the present disclosure are merely exemplary.

As used herein, terms such as “comprise”, “include”, “have”, “contain”, etc., are open-ended terms, meaning “including but not limited to”.

In the present disclosure:
6-BA is 6-benzylaminopurine having a molecular formula of C₁₂H₁₁N₅. CAS Registry Number 1214-39-7.
KT is kinetin, also known as 6-furfurylaminopurine having a molecular formula of C₁₀H₉N₅O. CAS Registry Number 525-79-1.
NAA is 1-naphthaleneacetic acid having a molecular formula of C₁₂H₁₀O₂. CAS Registry Number 86-87-3.
TDZ is thidiazuron having a molecular formula of C₉H₈N₄OS. CAS Registry Number 51707-55-2.
IBA is 3-indolebutyric acid having a molecular formula of C₁₂H₁₃NO₂. CAS Registry Number 133-32-4.
GA₃ is gibberellin having a molecular formula of C₁₉H₂₂O₆. CAS Registry Number 77-06-5.

### Example 1

### 1. Sterile Seedlings

Tender stems with buds of Emmenopterys henryi seedlings were used as explants. After disinfection, they were inoculated into MS+0.1 mg/L NAA+2.0 mg/L 6-BA+2.0 mg/L KT medium for initiation culture to obtain Emmenopterys henryi sterile seedlings. The specific experiment was as follows:

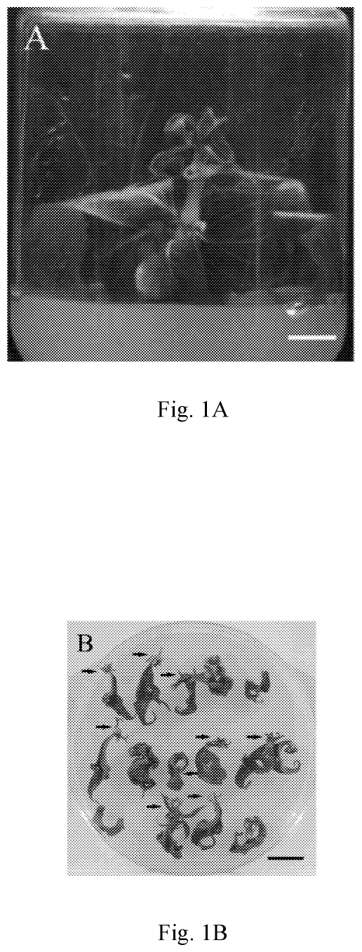

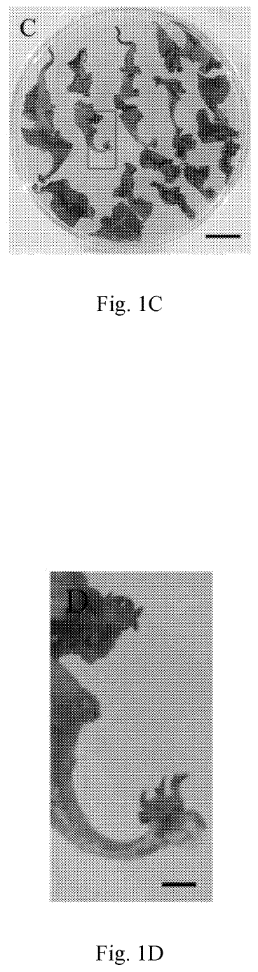

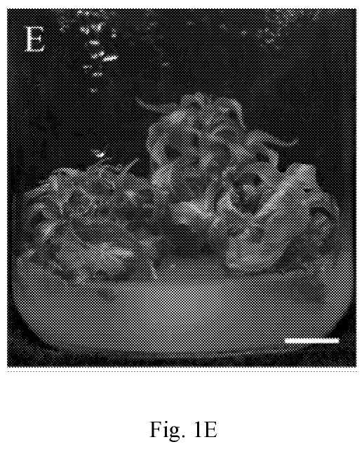

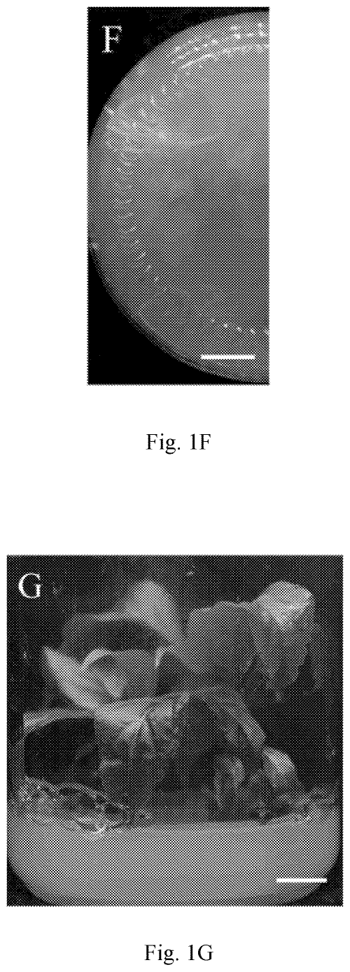

In March-April, tender stems with buds (terminal buds or axillary buds) of Emmenopterys henryi seedlings were used. The tender stems were cut into 5-7 cm long stem segments and rinsed under running water for 1-2 hours. In an ultra-clean workbench, the explants were soaked in 75% alcohol for 45 seconds, rinsed with sterile water 1-2 times, then soaked in 0.1% mercuric chloride (HgCl₂) solution for 6-8 minutes, and rinsed with sterile water 5 times. The stem segments were taken out and placed on sterile filter paper to absorb surface moisture. The two ends of the stem segments were cut off using a sterile scalpel (except for terminal buds), retaining 1-2 buds, and then were inoculated into a medium containing MS+0.1 mg/L NAA+2.0 mg/L 6-BA+2.0 mg/L KT+30 g/L sucrose+7.5 g/L agar and placed in a culture room for cultivation, with culture conditions: temperature of 24±2° C., light of 16 hours per day, light intensity of 100 μmol photon/m²/s (same culture room conditions apply hereafter). New tender leaves emerged from the explants on the 7th day, and the stems significantly elongated by the 30th day (FIG. 1A). The elongated stems were cut into segments, each retaining one terminal or axillary bud, for subsequent subculture.

2. Direct Induction of Adventitious Buds from Sterile Seedling Leaves

Leaves of 1-2 cm² from Emmenopterys henryi sterile seedlings were inoculated into 9 different MS media containing varying combinations of 6-BA and NAA hormones to induce the production of adventitious buds (Table 1). To further optimize the adventitious bud induction medium, TDZ (at concentration gradients of 0, 0.01, 0.1, 0.5, and 1.0 mg/L) was additionally added to the optimal differentiation medium (containing 1.0 mg/L 6-BA and 0.1 mg/L NAA) identified in the previous step (Table 2) to improve the adventitious bud induction rate. The optimal medium formula for direct induction of adventitious buds from sterile seedling leaves was: MS+1.0 mg/L 6-BA+0.1 mg/L NAA+0.01 mg/L TDZ+30 g/L sucrose+7.5 g/L agar. The specific experiment was as follows:

### (1) Induction of Leaf Adventitious Buds by 6-BA and NAA Combination

Leaves of 1-2 cm² from Emmenopterys henryi sterile seedlings were inoculated into 9 different MS media containing varying combinations of 6-BA and NAA hormones to induce adventitious buds (Table 1). Ten leaves were inoculated per petri dish (20 mm×90 mm), with 3 wounds cut on each leaf. During inoculation, the lower epidermis of the leaf was placed in close contact with the medium. Twenty leaves were inoculated for each treatment, with 3 replicates. After sealing, they were placed in the culture room. Photographs were taken every 10 days for recording. On the 30th day, the number of induced adventitious buds, the quantity of induced callus, and the state of the callus were statistically analyzed. The results showed that in Treatment No. 5 (containing 0.1 mg/L NAA+1.0 mg/L 6-BA), the leaves began to differentiate on the 10th day, produced adventitious buds on the 20th day, and developed numerous adventitious buds at the wound sites on the 30th day (FIG. 1B). In addition to adventitious buds, callus also formed at the wound sites across all treatments. The callus exhibited different states such as loose, compact, or browning. Loose callus appeared light yellow, while compact callus was emerald green. ANOVA was used to analyze the significance of differences in adventitious bud induction rate and callus induction rate among treatments. As shown in Table 1, the adventitious bud induction rate of leaves in Treatment No. 5 was significantly different from other treatments, while there was no significant difference in callus induction rate and callus state distribution. Treatment No. 5 had the highest efficiency in inducing adventitious buds from leaves, with an induction rate of 78.81%, and both the callus and adventitious buds were in good condition.

TABLE 1
Effects of 6-BA and NAA on Inducing Leaf Adventitious Buds and Callus

| Treatment | NAA | 6-BA | Callus Induction | Callus State Distribution (%) | | Adventitious Bud |
|---|---|---|---|---|---|---|
| No. | (mg/L) | (mg/L) | Rate (%) | Loose | Compact | Induction Rate (%) |
| 1 | 0.01 | 0.5 | 80.8 ± 6.9 | 23.3 ± 21.3 | 46.1 ± 7.2 | 7.3 ± 2.0d |
| 2 | 0.01 | 1 | 78.9 ± 17.7 | 16.8 ± 12.3 | 61.1 ± 13.2 | 14.4 ± 3.1cd |
| 3 | 0.01 | 2 | 81.7 ± 13.2 | 30.2 ± 27.3 | 61.0 ± 39.6 | 31.1 ± 22.0bc |
| 4 | 0.1 | 0.5 | 95.9 ± 3.8 | 30.9 ± 16.2 | 55.8 ± 4.1 | 14.3 ± 7.1cd |
| 5 | 0.1 | 1 | 96.7 ± 4.7 | 15.8 ± 20.3 | 78.2 ± 19.9 | 78.8 ± 9.2a |
| 6 | 0.1 | 2 | 91.3 ± 8.4 | 22.3 ± 11.1 | 77.7 ± 11.1 | 37.8 ± 16.2b |
| 7 | 1 | 0.5 | 90 ± 8.2 | 44.6 ± 29.4 | 44.1 ± 26.7 | 2.2 ± 3.1d |
| 8 | 1 | 1 | 68.9 ± 18.4 | 36.2 ± 38.4 | 62.0 ± 36.9 | 9.7 ± 2.4cd |
| 9 | 1 | 2 | 73.3 ± 37.7 | 32.2 ± 34.5 | 61.1 ± 43.7 | 5.0 ± 2.4d |

Note:
Values in columns are mean ± standard deviation.
Different letters after values indicate significant difference (P < 0.05).

### (2) Induction of Adventitious Buds Using 6-BA, NAA, and TDZ Combination

To optimize the medium formulation for inducing adventitious buds from leaves, TDZ was additionally added at concentrations of 0, 0.01, 0.1, 0.5, 1.0 mg/L to the optimal adventitious bud differentiation medium identified earlier (containing 0.1 mg/L NAA+1.0 mg/L 6-BA). For each treatment, 20 leaves were inoculated with three replicates. Three wounds were cut on each leaf, and the lower epidermis was placed in contact with the medium. After sealing, they were placed in the culture room. Photographs were taken every 10 days for recording. On the 30th day, the number of induced adventitious buds, induced callus, and callus state (loose, compact, or browning) were statistically analyzed. ANOVA was used to analyze the significance of differences in adventitious bud induction rate and callus induction rate among the treatments. After three repeated experiments, it was found that the medium with 0.01 mg/L TDZ (Treatment No. 2) resulted in a significantly higher adventitious bud induction rate than that of the control group without TDZ (Table 2), reaching 81.7%. Moreover, the adventitious buds were predominantly clustered shoots (FIGS. 1C and D), accounting for 74.2% of the total (Table 2), whereas the adventitious buds induced in Treatment No. 1 (without TDZ) were mostly single adventitious buds (FIG. 1B).

TABLE 2
Effects of 6-BA, NAA, and TDZ on Inducing Leaf Adventitious Buds and Callus

| | | Callus | | | Adventitious | Proportion of |
|---|---|---|---|---|---|---|
| Treatment | TDZ | Induction | Callus State Distribution (%) | | Bud Induction | Clustered |
| No. | (mg/L) | Rate (%) | Loose | Compact | Rate (%) | Buds (%) |
| 1 | 0 | 95.0 ± 4.1 | 57.9 ± 11.2 | 40.4 ± 13.6 | 63.5 ± 6.0b | 0b |
| 2 | 0.01 | 98.3 ± 2.4 | 18.5 ± 5.7 | 75.2 ± 13.5 | 81.7 ± 10.3a | 74.2 ± 11.0a |
| 3 | 0.1 | 95.6 ± 4.2 | 44.8 ± 7.3 | 50.5 ± 7.6 | 15.6 ± 10.3c | 3.7 ± 5.2b |
| 4 | 0.5 | 85.6 ± 10.3 | 53.0 ± 26.6 | 42.8 ± 28.0 | 25.6 ± 17.5c | 10.0 ± 14.1b |
| 5 | 1 | 97.8 ± 3.1 | 48.7 ± 27.3 | 47.7 ± 22.3 | 19.2 ± 9.0c | 8.3 ± 11.8b |

Note:
Values in columns are mean ± standard deviation.
Different letters after values indicate significant difference (P < 0.05).

3. Proliferation of Emmenopterys henryi Adventitious Buds

Adventitious buds of 1-2 cm in height were inoculated into 9 different MS media containing varying concentration combinations of 6-BA and IBA (Table 3) for proliferation culture. In the proliferation experiment, the 6-BA concentration was gradually increased (2.0, 3.0, 4.0, 5.0, 6.0 mg/L, respectively) (Table 4) to improve proliferation efficiency. The optimal medium formula for adventitious bud proliferation was: MS+3.0 mg/L 6-BA+0.1 mg/L IBA+30 g/L sucrose+7.5 g/L agar. The specific experiment was as follows:

(1) Effects of 6-BA and IBA on Emmenopterys henryi Adventitious Bud Proliferation

Adventitious buds of 1-2 cm in height were inoculated into 9 different MS media containing varying concentration combinations of 6-BA and IBA (Table 3) for proliferation culture. For each treatment, 30 adventitious buds were inoculated with 3 replicates. After 40 days of culture in the culture room, the number of proliferated adventitious buds and the state of new adventitious buds were observed, counted, and photographed. ANOVA was used to analyze the significance of differences in adventitious bud proliferation coefficients among treatments. As shown in Table 3, Treatment No. 7 (containing 2.0 mg/L 6-BA+0.1 mg/L IBA) has the highest proliferation coefficient of 3.9, and good adventitious bud state (FIG. 1E). Among these, adventitious buds with height>10 mm accounted for 24.1%, and those with height of 5-10 mm accounted for 48.3% (Table 3). Except for Treatment No. 4 (containing 1.5 mg/L 6-BA+0.1 mg/L IBA), the proliferation coefficient of Treatment No. 7 was significantly higher than that of other treatments.

TABLE 3
Effects of 6-BA and IBA on Adventitious Bud Proliferation

| | | | Adventitious Bud | | | |
|---|---|---|---|---|---|---|
| Treatment | 6-BA | IBA | Proliferation | Height Distribution of Adventitious Buds (%) | | |
| No. | (mg/L) | (mg/L) | Coefficient (%) | 2-5 mm | 5-10 mm | >10 mm |
| 1 | 1 | 0.1 | 2.8 ± 0.25bc | 34.8 ± 2.77 | 42.5 ± 5.11 | 22.7 ± 5.31 |
| 2 | 1 | 0.5 | 2.7 ± 0.4bcd | 26.8 ± 8.16 | 45.9 ± 8.47 | 27.3 ± 9.4 |
| 3 | 1 | 1 | 2.1 ± 0.4cd | 21.8 ± 5.54 | 40.7 ± 9.94 | 37.4 ± 7.22 |
| 4 | 1.5 | 0.1 | 2.9 ± 0.39b | 36.5 ± 9.58 | 47.7 ± 8.22 | 15.9 ± 8.79 |
| 5 | 1.5 | 0.5 | 2.7 ± 0.04bcd | 29.5 ± 8.42 | 36.8 ± 8.86 | 33.7 ± 3.76 |
| 6 | 1.5 | 1 | 2 ± 0.26d | 18.1 ± 2.98 | 45.5 ± 9.77 | 36.4 ± 9.97 |
| 7 | 2 | 0.1 | 3.9 ± 0.31a | 27.7 ± 9.76 | 48.3 ± 7.29 | 24.1 ± 4.31 |
| 8 | 2 | 0.5 | 2.5 ± 0.36bcd | 20.2 ± 9.76 | 52.3 ± 5.82 | 27.6 ± 4.35 |
| 9 | 2 | 1 | 2.4 ± 0.32bcd | 16.3 ± 8.46 | 51.8 ± 6.05 | 31.9 ± 3.56 |

Note:
Values in columns are mean ± standard deviation.
Different letters after values indicate significant difference (P < 0.05).

### (2) Optimization of Medium Formula for Adventitious Bud Proliferation

To further optimize the medium formula for adventitious bud proliferation, the 6-BA concentration was gradually increased (2.0, 3.0, 4.0, 5.0, 6.0 mg/L, respectively) (Table 4) based on the proliferation experiment. Adventitious buds of 1-2 cm in height were inoculated into MS media containing 6-BA (2.0, 3.0, 4.0, 5.0, 6.0 mg/L, respectively) and 0.1 mg/L IBA. For each treatment, 30 adventitious buds were inoculated with 3 replicates. After 40 days of culture, the number of proliferated adventitious buds and the state of new adventitious buds were counted and photographed. ANOVA was used for significance analysis in adventitious bud proliferation coefficient among treatments. As seen in Table 4, when the 6-BA concentration increased to 3.0 mg/L (Treatment No. 2), the adventitious bud proliferation coefficient was the largest and significantly higher than other treatments. When increased to 4.0 mg/L (Treatment No. 3), the proliferation coefficient decreased (Table 4), indicating that 3.0 mg/L 6-BA is the optimal concentration for adventitious bud proliferation.

TABLE 4
Optimization of 6-BA and IBA for Promoting Adventitious Bud Proliferation

| | | | Adventitious Bud | | | |
|---|---|---|---|---|---|---|
| Treatment | 6-BA | IBA | Proliferation | Height Distribution of Adventitious Buds (%) | | |
| No. | (mg/L) | (mg/L) | Coefficient (%) | 2-5 mm | 5-10 mm | >10 mm |
| 1 | 2 | 0.1 | 3.3 ± 0.3b | 13.8 ± 2.4b | 59.5 ± 1.4a | 26.7 ± 1.1 |
| 2 | 3 | 0.1 | 4.7 ± 0.5a | 27.5 ± 6.5a | 49.0 ± 8.2b | 23.5 ± 3.5 |
| 3 | 4 | 0.1 | 3.4 ± 0.4b | 27.0 ± 4.6a | 48.4 ± 4.1b | 24.6 ± 5.2 |
| 4 | 5 | 0.1 | 2.8 ± 0.3b | 26.1 ± 8.4ab | 46.3 ± 1.5b | 27.7 ± 7.1 |
| 5 | 6 | 0.1 | 2.7 ± 0.9b | 23.5 ± 3.9ab | 45.4 ± 1.6b | 31.2 ± 3.6 |

Note:
Values in columns are mean ± standard deviation.
Different letters after values indicate significant difference (P < 0.05).

4. Gibberellin Promoting Elongation of Emmenopterys henryi Adventitious Buds

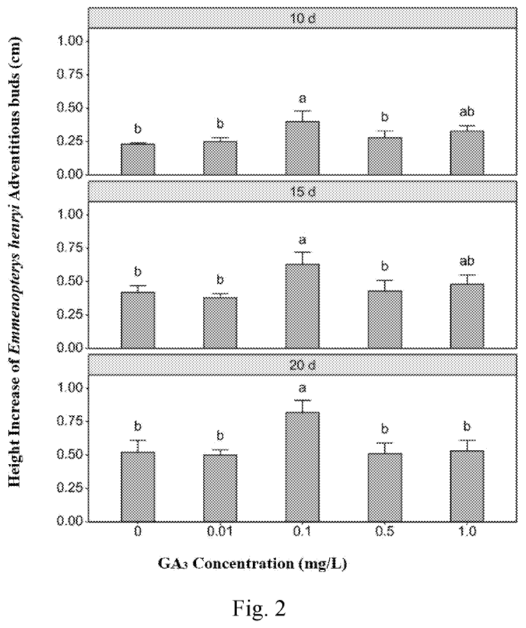

The Emmenopterys henryi adventitious buds obtained from proliferation culture were stripped of leaves, retaining only the terminal buds which were then inoculated into the optimal bud proliferation medium supplemented with GA₃ (concentration gradient of 0, 0.01, 0.1, 0.5, 1.0 mg/L) (FIG. 2) to promote adventitious bud elongation. The optimal medium formula for gibberellin-promoted adventitious bud elongation was: MS+3.0 mg/L 6-BA+0.1 mg/L IBA+0.1 mg/L GA₃+30 g/L sucrose+7.5 g/L agar. The specific experiment was as follows:

Using adventitious buds grown on the optimal proliferation medium (containing 3.0 mg/L 6-BA and 0.1 mg/L IBA) for Emmenopterys henryi as the material, the height of each adventitious bud was measured, and leaves were removed, retaining only the terminal bud. GA₃ (concentrations of 0, 0.01, 0.1, 0.5, 1.0 mg/L, respectively) was additionally added to the optimal proliferation medium. The treated adventitious buds were inoculated onto these media. For each treatment, 30 adventitious buds were inoculated, with each bud labeled on the culture vessel with its serial number and initial height. The culture vessels were placed in the tissue culture room. On the 10th, 15th, and 20th days, the height growth of the adventitious buds was measured and recorded. As shown in FIG. 2, as the concentration of GA₃ increased, the change in adventitious bud height showed a trend of first increasing and then decreasing. The elongation of buds treated with 0.1 mg/L GA₃ exhibited the most significant difference compared to the control group. On the 10th day of culture, the difference in height change compared to the control group was the greatest (FIG. 2). By day 20, the increase in bud height was significantly greater in the 0.1 mg/L GA₃ treatment than in the control and other concentration treatments, indicating that 0.1 mg/L GA₃ has a significant effect on promoting the elongation of Emmenopterys henryi adventitious buds.

5. Rooting Induction of Emmenopterys henryi Adventitious Buds

Adventitious buds of 2-3 cm in length were used and inoculated into ½ MS and MS media containing IAA (0, 0.5, 1.0, 2.0 mg/L), IBA (0, 0.5, 1.0, 2.0 mg/L), or NAA (0.5, 1.0, 2.0 mg/L) (Table 5) to induce rooting. The optimal medium formula for adventitious bud rooting was: ½ MS+1.0 mg/L IBA+30 g/L sucrose+7.5 g/L agar. The specific experiment was as follows:

Two adventitious buds were inoculated per culture vessel, with leaves below the terminal buds removed. After 40 days of culture in the tissue culture room, the number of rooted adventitious buds and the number of roots were counted and photographed. ANOVA was used for significance analysis of differences in root number and rooting rate among treatments. Compared with full-strength MS, ½ MS yielded better rooting induction, and Treatment No. 13 in ½ MS medium (IBA concentration 1.0 mg/L) had the highest rooting rate of 77.8%, producing 1 to 3 roots (Table 5, FIG. 1F). The rooting rate induced by full-strength MS medium was lower. Although the tissue culture plantlets induced by Treatment No. 3 (IBA concentration 1.0 mg/L) were in good condition with thicker stems and roots, the roots were shorter, and the rooting rate was only 26.2% (Table 5).

TABLE 5
Induction of Adventitious Bud Rooting Using IAA, IBA, and NAA

| | Treatment | IBA | NAA | IAA | | |
|---|---|---|---|---|---|---|
| Medium | No. | (mg/L) | (mg/L) | (mg/L) | Number of Roots | Rooting Rate (%) |
| MS | 1 | 0 | 0 | 0 | 0e | 0d |
| MS | 2 | 0.5 | 0 | 0 | 1.3 ± 1.2abcde | 9.5 ± 6.7cd |
| MS | 3 | 1 | 0 | 0 | 1.5 ± 0.4abcde | 26.2 ± 16.8bcd |
| MS | 4 | 2 | 0 | 0 | 0.3 ± 0.5e | 5.6 ± 7.9d |
| MS | 5 | 0 | 0.5 | 0 | 0.7 ± 0.9cde | 4.8 ± 6.7 d |
| MS | 6 | 0 | 1 | 0 | 0e | 0d |
| MS | 7 | 0 | 2 | 0 | 0e | 4.8 ± 6.7d |
| MS | 8 | 0 | 0 | 0.5 | 0e | 0d |
| MS | 9 | 0 | 0 | 1 | 0.5 ± 0.7cde | 11.1 ± 15.7cd |
| MS | 10 | 0 | 0 | 2 | 0e | 0d |
| ½ MS | 11 | 0 | 0 | 0 | 0e | 0d |
| ½ MS | 12 | 0.5 | 0 | 0 | 2.7 ± 1.6a | 58.3 ± 21.1ab |
| ½ MS | 13 | 1 | 0 | 0 | 1.9 ± 0.4abcd | 77.8 ± 7.9a |
| ½ MS | 14 | 2 | 0 | 0 | 2.4 ± 0.6ab | 55.6 ± 28.3ab |
| ½ MS | 15 | 0 | 0.5 | 0 | 1.2 ± 0.3abcde | 42.3 ± 40.8bc |
| ½ MS | 16 | 0 | 1 | 0 | 2.3 ± 0.5ab | 56.0 ± 21.9ab |
| ½ MS | 17 | 0 | 2 | 0 | 0e | 4.2 ± 5.9d |
| ½ MS | 18 | 0 | 0 | 0.5 | 1.2 ± 1.0abcde | 14.3 ± 11.7cd |
| ½ MS | 19 | 0 | 0 | 1 | 0.8 ± 0.6bcde | 13.7 ± 11.7cd |
| ½ MS | 20 | 0 | 0 | 2 | 2 ± 0.8 abc | 18.5 ± 7.2 cd |

Note:
Values in columns are mean ± standard deviation.
Different letters after values indicate significant difference (P < 0.05).

6. Hardening and Transplanting of Emmenopterys henryi Tissue Culture Plantlets

In spring or early summer, two-month-old rooted Emmenopterys henryi tissue culture plantlets were planted in two-color pots containing a mixed substrate of peat soil:substrate soil:vermiculite (mass ratio 3:3:1). The Emmenopterys henryi tissue culture plantlets retained only the leaves of the second stem node, with other leaves removed. The root system was gently buried in the substrate, thoroughly watered several times, covered with a transparent lid, and placed in the experimental base for cultivation. They were watered twice a day (morning and evening) for two weeks, and thereafter three times a week. The survival rate was 100% after 40 days of transplanting.

The embodiments described above are merely preferred modes of the present disclosure and do not limit the scope of the present disclosure. Various modifications and improvements made by those of ordinary skill in the art without departing from the spirit of the present disclosure shall fall within the protection scope defined by the claims.

What is claimed is:

1. A method for efficient and rapid propagation of Emmenopterys henryi, comprising the following steps:
culturing a tender stem with buds from an Emmenopterys henryi seedling as an explant on an initiation medium to obtain a sterile seedling;
taking leaves of the sterile seedling and inducing differentiation of the leaves on a differentiation medium to obtain adventitious buds;
subjecting the adventitious buds to proliferation culture on a proliferation medium to obtain proliferated adventitious buds;
culturing the proliferated adventitious buds on an elongation medium to obtain elongated adventitious buds;
subjecting the elongated adventitious buds to rooting culture on a rooting medium to obtain tissue culture plantlets; and
transplanting the tissue culture plantlets into a mixed substrate for hardening,
wherein the differentiation medium is Murashige and Skoog (MS) medium containing 1.0 mg/L 6-benzylaminopurine (6-BA), 0.1 mg/L 1-naphthaleneacetic acid (NAA), and 0.01 mg/L thidiazuron (TDZ); and
the elongation medium is MS medium containing 3.0 mg/L 6-BA, 0.1 mg/L indole-3-butyric acid (IBA), and 0.1 mg/L gibberellic acid (GA₃).

2. The method according to claim 1, wherein the initiation medium is MS medium containing 2.0 mg/L 6-BA, 2.0 mg/L kinetin (KT), and 0.1 mg/L NAA.

3. The method according to claim 1, wherein the proliferation medium is MS medium containing 3.0 mg/L 6-BA and 0.1 mg/L IBA.

4. The method according to claim 1, wherein prior to the elongation culture, the method further comprises a step of removing leaves from the proliferated adventitious buds and retaining only terminal buds.

5. The method according to claim 1, wherein the rooting medium is ½ MS medium containing 1.0 mg/L IBA.

6. The method according to claim 1, wherein the mixed substrate is composed of peat soil, substrate soil, and vermiculite, wherein a mass ratio of the peat soil, the substrate soil, and the vermiculite is 3:3:1.

7. A culture medium combination for Emmenopterys henryi, comprising an initiation medium, a differentiation medium, a proliferation medium, an elongation medium, and a rooting medium;
wherein the initiation medium is MS medium containing 2.0 mg/L 6-BA, 2.0 mg/L KT, and 0.1 mg/L NAA;
the differentiation medium is MS medium containing 1.0 mg/L 6-BA, 0.1 mg/L NAA, and 0.01 mg/L TDZ;
the proliferation medium is MS medium containing 3.0 mg/L 6-BA and 0.1 mg/L IBA;
the elongation medium is MS medium containing 3.0 mg/L 6-BA, 0.1 mg/L IBA, and 0.1 mg/L GA₃; and
the rooting medium is ½ MS medium containing 1.0 mg/L IBA.

8. Application of the culture medium combination according to claim 7 in the propagation of Emmenopterys henryi.
